(19) United States
(12) Patent Application Publication
(10) Pub. No.: US 2012/0136279 A1
(43) Pub. Date: May 31, 2012

(54) ULTRASOUND SURGICAL APPARATUS

(75) Inventors: Kazue TANAKA, Sagamihara-shi (JP); Yukihiko SAWADA, Yoshikawa-shi (JP); Norihiro YAMADA, Tokyo (JP); Takashi MIHORI, Tokyo (JP)

(73) Assignee: OLYMPUS MEDICAL SYSTEMS CORP., Tokyo (JP)

(21) Appl. No.: 13/307,966

(22) Filed: Nov. 30, 2011

Related U.S. Application Data
(63) Continuation of application No. PCT/JP2009/062315, filed on Jul. 6, 2009.

(51) Int. Cl. A61N 7/00

(52) U.S. Cl. 601/2

(57) ABSTRACT

An ultrasound surgical apparatus includes: an ultrasound transducer that generates ultrasound vibrations; a driving portion that supplies a driving signal to the ultrasound transducer; a distal end portion that is mechanically coupled with the ultrasound transducer and treats a living tissue through a liquid; a detecting portion that detects a cavitation level signal corresponding to a state of cavitation generated in the liquid by ultrasound vibrations of the distal end portion; and an information acquiring portion that acquires information of the living tissue on the basis of the cavitation level signal.

### CROSS REFERENCE TO RELATED APPLICATION

This application is a continuation application of PCT/JP2009/062315 filed on Jul. 6, 2009, the entire contents of which are incorporated herein by this reference.

### BACKGROUND OF THE INVENTION

1. Field of the Invention

The present invention relates to an ultrasound surgical apparatus for treating living tissues.

2. Description of the Related Art

Examples of ultrasound surgical apparatuses that treat living tissues using ultrasound vibrations include ultrasound coagulating/cutting apparatuses, ultrasound suction apparatuses, ultrasound lithotripsy apparatuses, and ultrasound trocars.

An ultrasound coagulating/cutting apparatus grasps a tissue with an ultrasound-vibrating probe, thereby generating friction heat to carry out coagulation or dissection processing. Ultrasound coagulating/cutting apparatuses can perform low-temperature processing compared with electrical surgical apparatuses, resulting in minor damage of tissues. With a probe on which high-frequency waves can be applied, a styptic treatment can be easily performed.

Ultrasound suction apparatuses take advantage of tissue selectivity of ultrasound to emulsify and suck only a weak tissue using ultrasound vibrations and can exposure elastic tissues such as blood vessels without destroying them.

An ultrasound lithotripsy apparatus brings a probe vibrating with ultrasound directly into a calculus or the like to change the ultrasound vibrations into an impact, thereby crushing the calculus.

In ultrasound surgical apparatuses that carry out treatment using cavitation generated by ultrasound vibrations, a state of the cavitation is significant. For example, International Publication No. 2005/094701 discloses an ultrasound applying method by which a state of cavitation is detected from a sound pressure signal in order to maintain a predetermined cavitation state.

For ultrasound surgical apparatuses, information of a tissue to be treated is significant, and there is, in particular, a need for an ultrasound surgical apparatus that acquires information of a tissue during the treatment.

### SUMMARY OF THE INVENTION

An ultrasound surgical apparatus of an embodiment of the present invention includes: an ultrasound transducer that generates ultrasound vibrations; a driving portion that supplies a driving signal to the ultrasound transducer; a treating portion that is mechanically coupled with the ultrasound transducer and treats a living tissue through a liquid; a detecting portion that detects a cavitation level signal corresponding to a state of cavitation generated in the liquid by ultrasound vibrations of the treating portion; and an information acquiring portion that acquires information of the living tissue on the basis of the cavitation level signal.

### BRIEF DESCRIPTION OF THE DRAWINGS

FIG. 1 is a block diagram of an ultrasound surgical apparatus of a first embodiment.

FIG. 2 is a diagram for explaining a cavitation generation mechanism in accordance with the ultrasound surgical apparatus of the first embodiment.

FIG. 3 is a diagram for explaining the cavitation generation mechanism in accordance with the ultrasound surgical apparatus of the first embodiment.

FIG. 4 is a diagram for describing a cavitation level signal appearing when cavitation is not being generated in accordance with the ultrasound surgical apparatus of the first embodiment.

FIG. 5 is a diagram for describing a cavitation level signal appearing when cavitation is being generated in accordance with the ultrasound surgical apparatus of the first embodiment.

FIG. 6 is a diagram for describing a driving signal from the ultrasound surgical apparatus of the first embodiment.

FIG. 7 is a diagram for describing a cavitation level signal in accordance with the ultrasound surgical apparatus of the first embodiment.

FIG. 8 is a diagram for describing a cavitation level signal in accordance with the ultrasound surgical apparatus of the first embodiment.

FIG. 9 is a diagram showing a relationship between a pace of attenuation of a cavitation level signal and hardness in accordance with the ultrasound surgical apparatus of the first embodiment.

FIG. 10 is a diagram for explaining a state in which cavitation is generated in accordance with an ultrasound surgical apparatus of a second embodiment.

FIG. 11 is a diagram for explaining a state in which cavitation is generated in accordance with the ultrasound surgical apparatus of the second embodiment.

FIG. 12 is a diagram showing a relationship between a cavitation level signal and a distance D from a distal end portion to a blood vessel wall in accordance with the ultrasound surgical apparatus of the second embodiment.

FIG. 13 is an appearance view for explaining a configuration of an ultrasound surgical apparatus of a third embodiment.

FIG. 14 is a schematic diagram for explaining a configuration of a probe of the ultrasound surgical apparatus of the third embodiment.

FIG. 15 is a block diagram of the ultrasound surgical apparatus of the third embodiment.

FIG. 16 is a diagram for describing a relationship between a time course and electric resistance of a tissue in the treatment carried out in accordance with the ultrasound surgical apparatus of the third embodiment.

### DETAILED DESCRIPTION OF THE PREFERRED EMBODIMENTS

### First Embodiment

Now, an ultrasound surgical apparatus 1 of a first embodiment of the present invention will be described with reference to the drawings.

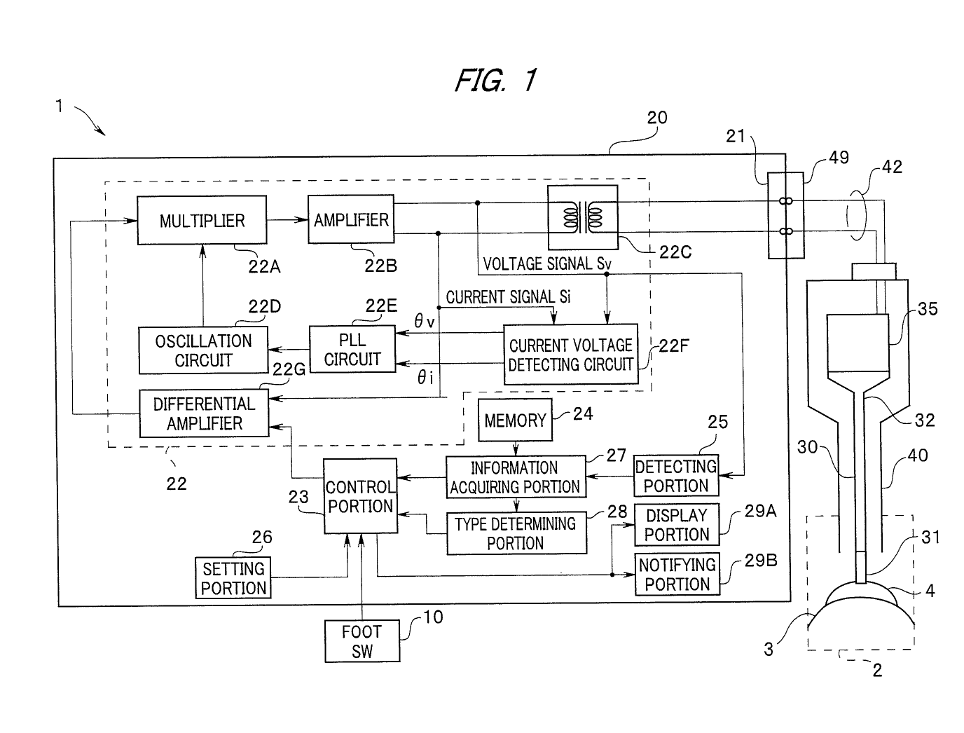

As shown in FIG. 1, the ultrasound surgical apparatus 1 of the present embodiment is a suction-type ultrasound surgical apparatus including an apparatus main body portion 20, an ultrasound suction-type handpiece 40 connected with a socket 21 of the apparatus main body portion 20 through a connector 49 using cables 42, and a foot switch 10 connected with the apparatus main body portion 20.

The handpiece 40 includes an ultrasound transducer (hereinafter, also referred to as the “transducer”) 35 and a cylindrical column shaped probe 30, a proximal end portion 32 of which is mechanically coupled to the transducer 35. The probe 30 transfers vibrations generated by the transducer 35 to a distal end portion 31 of the probe 30. The distal end portion 31 is a treating portion that treats a living tissue (hereinafter, also referred to as the “tissue”) 3 through a liquid 4 and may be detachable from the probe 30.

The apparatus main body portion 20 of the ultrasound surgical apparatus 1 includes a driving portion 22, a control portion 23, a setting portion 26, a detecting portion 25, an information acquiring portion 27, memory 24, a type determining portion 28, a display portion 29A, and a notifying portion 29B. Under the control of the control portion 23, the driving portion 22 outputs a current controlled driving signal for driving the transducer 35. For example, the control portion 23 being a CPU controls the entire ultrasound surgical apparatus 1 including the driving portion 22. It should be noted that in the ultrasound surgical apparatus 1, although the information acquiring portion 27 and the type determining portion 28 are separate components, the information acquiring portion 27 and the type determining portion 28 may be integrated with each other, and at least one of the portions 27 and 28 may be integrated with the control portion 23. In addition, the memory 24 may be integrated with the information acquiring portion 27 or the control portion 23.

As described later, the detecting portion 25 detects the cavitation level signal corresponding to a state of cavitation generated in the liquid 4 by ultrasound vibrations of the distal end portion 31. The information acquiring portion 27 acquires information of the tissue 3 based on the cavitation level signal detected by the detecting portion 25 and data stored in the memory 24. The type determining portion 28 determines a type of the tissue 3 on the basis of the information of the tissue 3 acquired by the information acquiring portion 27. The display portion 29A and the notifying portion 29B allow an operator to recognize an operating state of the apparatus main body portion 20 and the like, and also function as an alarm issuing portion that issues an alarm to the operator.

That is, the ultrasound surgical apparatus disclosed in International Publication No. 2005/094701, which is known, detects a state of cavitation and maintains a predetermined cavitation state, and the ultrasound surgical apparatus 1 of the present embodiment is similar to the known ultrasound surgical apparatus in that the ultrasound surgical apparatus 1 detects a state of cavitation. However, the ultrasound surgical apparatus 1 senses a state of cavitation based on a specific frequency component signal of the driving signal and acquires, in real time, information of the tissue 3 being treated, based on the cavitation level signal corresponding to the state of cavitation.

The driving portion 22 includes an oscillation circuit 22D, a multiplier 22A, an amplifier 22B, an output circuit 22C, a current voltage detecting circuit 22F, a PLL (Phase-Locked Loop) circuit 22E, and a differential amplifier 22G. An oscillation signal generated at the oscillation circuit 22D is inputted to the multiplier 22A, the signal multiplied at the multiplier 22A is amplified at the amplifier 22B, and the amplified signal is outputted through the output circuit 22C to the transducer 35. The output circuit 22C is formed of, for example, a transformer, and the driving signal amplified at the amplifier 22B is inputted to a primary winding side and a secondary winding side outputs the driving signal insulated from the driving signal of the primary winding side. Further, the primary windings of the output circuit 22C transformer are connected with the current voltage detecting circuit 22F in order to detect current of the driving signal that runs through the primary windings and voltage at both ends thereof as well as to detect a current phase and a voltage phase.

A current phase signal θi and a voltage phase signal θv detected by the current voltage detecting circuit 22F are outputted to the PLL circuit 22E. The PLL circuit 22E outputs to the oscillation circuit 22D a control signal, a signal level (signal strength) of which varies depending on a phase difference between the current phase signal θi and the voltage phase signal θv. The oscillation circuit 22D is, for example, a voltage controlled oscillator (VCO), an oscillation frequency of which varies depending upon a level of the inputted signal. The PLL circuit 22E outputs, to the oscillation circuit 22D, an oscillation frequency adjusting signal for reducing a phase difference between the current phase signal θi and the voltage phase signal θv. Thus, in the oscillation circuit 22D, an oscillation frequency is automatically adjusted to cause a phase difference between the current phase signal θi and the voltage phase signal θv to be 0 by a closed loop with the PLL circuit 22E. It is noted that the oscillation frequency in which a phase difference between the current phase signal θi and the voltage phase signal θv is 0 is a frequency corresponding to a resonance frequency, fres (e.g., 47 kHz), of the transducer 35. That is, the PLL circuit 22E automatically adjusts an oscillation frequency so as to drive the transducer 35 with the driving signal of the resonance frequency. The differential amplifier 22G causes a driving signal level to be an output value set by the setting portion 26 or the foot SW 10 or a value controlled by the control portion 23, as described later.

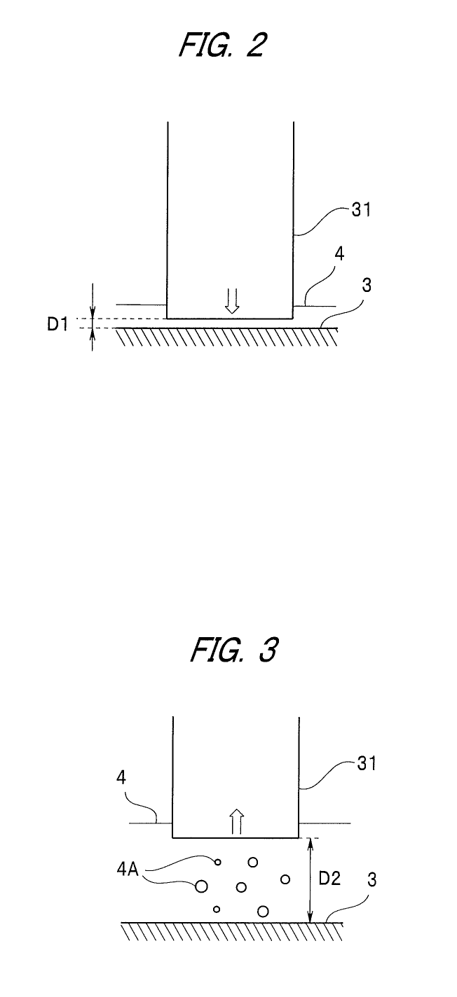

Next, an operation of the ultrasound surgical apparatus 1 will be described with reference to FIGS. 2 and 3. In treatment, the distal end portion 31 of the ultrasound surgical apparatus 1 is inserted in a living body 2 and placed close to the tissue 3 to be treated. It is noted that the liquid 4 exists between the distal end portion 31 and the tissue 3. The liquid 4 is a body fluid or water, such as Ringer's solution, supplied from a water supplying portion (not shown) of the probe 30. The distal end portion 31 alternates a state in which a distance to the tissue 3 is D1 (FIG. 2) and a state in which a distance to the tissue 3 is D2 (FIG. 3) by the ultrasound vibrations. Amplitude of the ultrasound vibrations is (D2-D1) and varies depending on the control signal level. If a distance (gap) between the distal end portion 31 and the tissue 3 shifts from the state in FIG. 2 to the state in FIG. 3, the liquid between the distal end portion 31 and the tissue 3 is put under a negative pressure, resulting in the generation of cavitation, that is, cavitation bubbles 4A are generated.

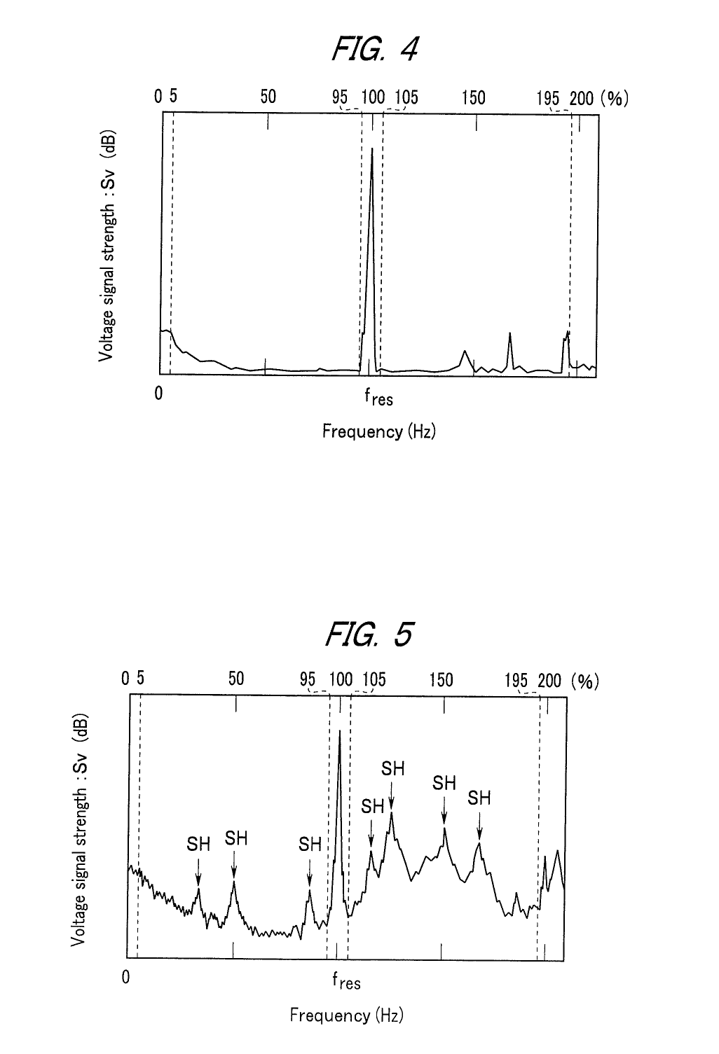

Now, FIG. 4 illustrates a frequency spectrum distribution of the voltage signal Sv of the driving signal appearing when cavitation is not being generated, and FIG. 5 illustrates a frequency spectrum distribution of the voltage signal Sv appearing when cavitation is being generated. It should be noted that in FIGS. 4 and 5, upper numbers mean frequencies with a resonance frequency fres as 100%.

As shown in FIG. 4, when cavitation is not being generated, the voltage signal Sv does not have prominent peaks at frequencies other than the resonance frequency fres. On the other hand, as shown in FIG. 5, when cavitation is being generated, the voltage signal Sv has higher levels than when cavitation is not being generated, at the frequencies other than the resonance frequency fres. That is, when cavitation is being generated, unlike when cavitation is not being generated, the voltage signal Sv has peaks of frequencies of subharmonics (SH), which are submultiples or differences of submultiples such as ½ or ¼ of the resonance frequency fres as well as levels at frequencies other than the subharmonics are also higher than when cavitation is not being generated. Then, as a state of cavitation becomes violent, gaps of levels of voltage signal Sv from the case where cavitation is not being generated become wider, that is, the levels become higher.

Thus, the detecting portion 25 can detect a state of cavitation by detecting the signal except that around the resonance frequency fres of the voltage signal Sv of the driving signal as the cavitation level signal. For example, as a cavitation level signal, the signal obtained by filtering the voltage signal Sv to acquire (integrate) only components of frequencies from 5% to 95% of the resonance frequencies fres can be preferably used. Alternatively, as a cavitation level signal, the signal obtained by filtering the voltage signal Sv to acquire components of frequencies from those higher than the resonance frequency fres by 5% to those lower than frequencies of second harmonics of the resonance frequency fres (2 fres) by 5% may also be used. Further, as a cavitation level signal, the signal obtained by acquiring frequency components except the frequency components around 5% above or below the resonance frequency fres may also be used. In addition, as a cavitation level signal, the signal obtained by acquiring frequency components of subharmonics (SH) of the voltage signal Sv or peak strengths may be preferably used.

It should be noted that the cavitation level signal is not limited to the voltage signal Sv, and may be an impedance signal or a current signal if the driving signal voltage controlled by the driving portion 22 is used.

Further, the cavitation level signal strength sensed by the sensing portion 25 vary depending on the various conditions such as the type of the liquid 4, the amplitude of the ultrasound vibrations, or the states of the tissue 3, but in the case of the same type of the liquid 4 and the same amplitude of the ultrasound vibrations, the cavitation level signal strength indicate the state of the tissue 3. Thus, the information acquiring portion 27 can acquire information of a water content of the tissue 3 based on cavitation level signal strength. That is, the cavitation level signal becomes higher in the case of a larger water content of the tissue 3 than in the case of a smaller water content of the tissue 3.

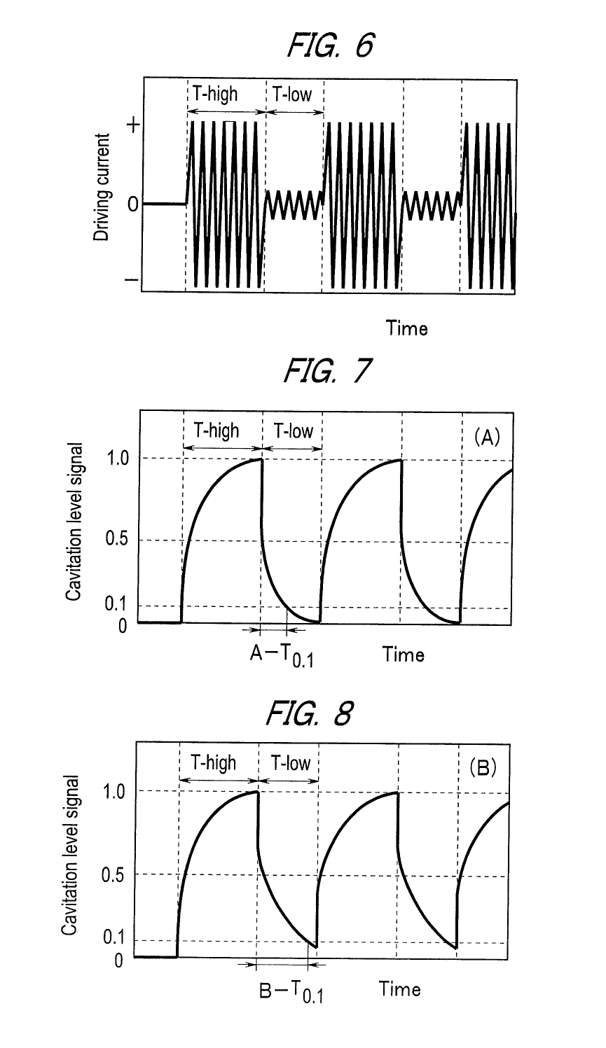

Then, as shown in FIG. 6, in the ultrasound surgical apparatus 1 of the present embodiment, the driving portion 22 alternates high output with low output of the signal strength of the driving signal to be supplied to the transducer 35 and outputs the driving signal. It is noted that the driving signal with high-output signal strength is signal strength in which cavitation for treatment is generated, while the driving signal with low-output signal strength is signal strength in which cavitation is not generated and signal strength for detecting the pace of attenuation of the cavitation level signal.

It is noted that high-output signal supply time T-high and low-output signal supply time T-low as shown in FIG. 6 are appropriately determined For example, the high-output signal supply time T-high is 10 ms to 10 seconds, and T-high/(T-high+T-low) is 0.5 to 0.99. If the high-output signal supply time T-high is at or above the range, cavitation having strengths sufficient for the detecting portion 25 to sense the cavitation level signal is generated, and if the time T-high is at or below the range, the information acquiring portion 27 can acquire information within time intervals required for treatment. Further, if T-high/(T-high+T-low) is at or above the range, the efficiency of treatment does not decrease, and if T-high/(T-high+T-low) is at or below the range, the accuracy of the information acquired by the information acquiring portion 27 does not decrease. It should be noted that waveforms in FIG. 6 are schematically illustrated.

It is noted that FIGS. 7 and 8 are associated with FIG. 6; FIG. 7 illustrates the cavitation level signal appearing while a lesional tissue A is being treated, and FIG. 8 illustrates the cavitation level signal appearing while a normal tissue B is being treated. It should be noted that the cavitation level signal is that obtained by acquiring (integrating) frequency components within the range of 5% to 95% of a resonance frequency fres of the constant-current driving voltage signal.

As shown in FIGS. 7 and 8, if the transducer 35 is supplied with the high-output driving signal, since cavitation bubbles 4A are generated, the cavitation level signal increases. However, if the driving signal shifts to the low-output signal, since cavitation bubbles 4A are not newly generated and going to burst, the cavitation level signal attenuates. It is noted that the pace of attenuation of the cavitation level signal is higher in the case of treating the tissue A (FIG. 7) than in the case of treating the tissue B (FIG. 8). It is due to the fact that the hardness Hv-A of the tissue A is higher than the hardness Hv-B of the tissue B.

That is, in the ultrasound surgical apparatus 1, the information of the tissue 3 can be acquired on the basis of the pace of attenuation of the cavitation level signal. It is noted that the ultrasound surgical apparatus 1 may use the pace of attenuation itself, or use the rate of attenuation. The rate of attenuation may be based on, for example, the time between the signal strength 1.0 at the cavitation level signal strength of immediately after shifting the driving signal and the signal strength 0.1 (10% attenuation time: T_{0.1}).

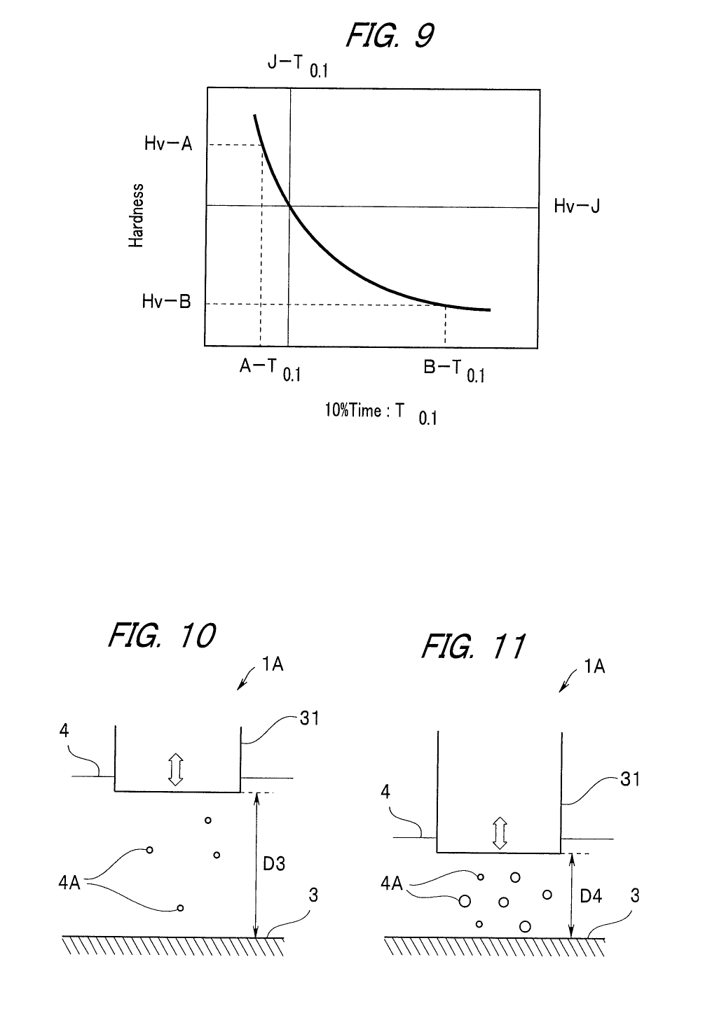

For example, as shown in FIG. 9, 10% attenuation time (T_{0.1}), namely, the rate of attenuation of the cavitation level signal is correlated with the hardness of the tissue 3 being treated, and 10% attenuation time (T_{0.1}) of the tissue A, A-T_{0.1}, is shorter than B-T_{0.1} of the tissue B. That is, if the pace of attenuation of the cavitation level signal is faster, the hardness of the tissue A, Hv-A, is higher than Hv-B of the tissue B. It is noted that because the hardness of the tissue 3 is inversely proportional to the water content of the tissue 3, hardness information is also water content information.

For example, in the ultrasound surgical apparatus 1, a relationship between the rate of attenuation of the cavitation level signal and hardness as shown in FIG. 9 is acquired in advance and stored in the memory 24, and thereby the information acquiring portion 27 acquires hardness information being information of the tissue 3 on the basis of the cavitation level signal detected by the detecting portion 25. In addition, the type determining portion 28 determines whether the tissue 3 is a normal tissue or a lesional tissue on the basis of the hardness of the tissue 3 acquired by the information acquiring portion 27. For example, as shown in FIG. 9, if the hardness is higher than predetermined hardness Hv-J, the type determining portion 28 determines that the tissue 3 is a lesional tissue. In addition, the determining portion 28 can also determine the type of the tissue 3, muscles, parenchymal organs or fatty tissues.

In the description made hereinbefore, the ultrasound surgical apparatus 1 has been described in which the driving portion 22 alternates high and low outputs of the signal strength of the driving signal supplied to the transducer 35 and outputs the driving signal, but the driving portion 22 may intermittently supply the transducer 35 with the driving signal. That is, once the transducer 35 stops vibrations, it may take time to start vibrations again, but in the case where the time lag causes no problem, the driving signal may be intermittently supplied.

If the driving signal is intermittently supplied, when the ultrasound transducer 35 does not vibrate, the detecting portion 25 detects burst sounds of cavitation bubbles 4A, the signal of subharmonic (SH) frequency components, and the like as the cavitation level signal by using the transducer 35 as a sensor. Then, the information of the tissue 3 is acquired from the pace of attenuation of the cavitation level signal.

The control portion 23 controls the signal strength of the driving signal supplied by the driving portion 22 in accordance with the information of the tissue 3 acquired by the information acquiring portion 27. That is, while a lesional tissue is being removed, in response to lowering of the pace of attenuation of the cavitation level signal, the information acquiring portion 27 notifies the control portion 23 that the lesional tissue has been removed and a normal tissue has been exposed. Then, the control portion 23 decreases the signal strength of the driving signal being the high-output signal supplied to the transducer 35. Thus, in the ultrasound surgical apparatus 1, a normal tissue can be prevented from being damaged. Also, the control portion 23 may display the cavitation level signal sensed by the detecting portion 25 or the information of the tissue 3 acquired by the information acquiring portion 27 on the display portion 29A. Furthermore, for example, when the control portion 23 decreases the signal strength of the driving signal on the basis of the information from the information acquiring portion 27, the control portion 23 may cause the display portion 29A and the notifying portion 29B, also functioning as an alarm issuing portion, to issue an alarm to the operator with characters, signs, voice, light or vibrations.

As hereinbefore described, the ultrasound surgical apparatus 1 provides high operability. Furthermore, the ultrasound surgical apparatus 1 offers a superior level of safety.

### Second Embodiment

Next, an ultrasound surgical apparatus 1A of a second embodiment of the present invention will be described. It should be noted that because the ultrasound surgical apparatus 1A of the present embodiment is similar to the ultrasound surgical apparatus 1 of the first embodiment, like components having the same functions are denoted by the same reference numbers and a description thereof is omitted.

An information acquiring portion of the ultrasound surgical apparatus 1A of the present embodiment acquires information of a distance D between a tissue 3 and a distal end portion 31 being a treating portion on the basis of the strength of a cavitation level signal. As described with reference to FIGS. 2 and 3, the cavitation generation mechanism of the ultrasound surgical apparatus 1A is vastly different from the mechanism in which cavitation is generated by ultrasound applied into liquid.

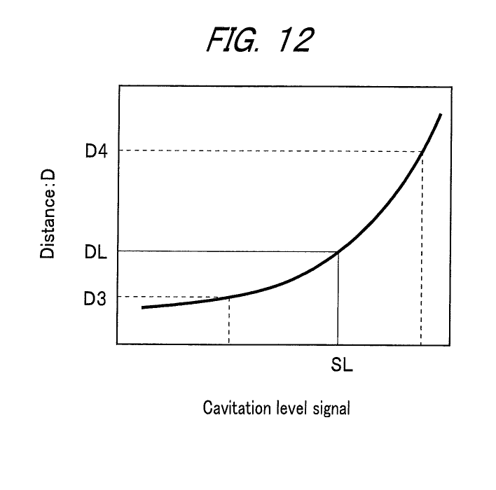

Therefore, as shown in FIGS. 10 and 11, as a distance D from the distal end portion 31 to the tissue 3 such as a blood vessel wall becomes shorter, higher negative pressure is generated in liquid by ultrasound vibrations, so that more cavitation bubbles 4A are generated. Thus, even if the distal end portion 31 is vibrating at the same amplitude, that is, at the same vibration strength, as shown in FIG. 12, in the ultrasound surgical apparatus 1A, the strength of the cavitation level signal is inversely proportional to the distance D.

Therefore, in the ultrasound surgical apparatus 1A, for example, the relationship between the strength of the cavitation level signal and the distances D as shown in FIG. 12 is acquired in advance and stored in the memory 24, and thereby the information acquiring portion 27 acquires the information of a distance from the tissue 3 to the distal end portion on the basis of the cavitation level signal detected by the detecting portion 25 and the data stored in the memory 24.

Furthermore, when the distal end portion 31 comes closer than a predetermined distance DL from the tissue 3, the control portion 23 of the ultrasound surgical apparatus 1A decreases the signal strength of the driving signal supplied to the transducer 35 from the driving portion 22. That is, if the cavitation level signal exceeds a predetermined strength SL, the information acquiring portion 27 acquires the information that the distal end portion 31 is closer than the predetermined distance DL from the tissue 3, and the control portion 23 controls the driving portion 22 on the basis of the information. Thus, in the ultrasound surgical apparatus 1A, blood vessels can be prevented from being damaged. It should be noted that if the control portion 23 senses that the distal end portion 31 which once came close is now at the predetermined distance DL or longer from the tissue 3, the control portion 23 may increase the signal strength again. Thus, the ultrasound surgical apparatus 1A provides high operability.

Further, in the ultrasound surgical apparatus 1A, for example, a distance D may be simply presented to an operator by changing the number of illuminating LEDs on the display portion 29A composed of a plurality of LEDs on the basis of the distance information acquired by the information acquiring portion 27. In addition, when the distal end portion 31 comes closer than a predetermined distance DL from the tissue 3, the control portion 23 may cause the display portion 29A and the notifying portion 29B, also functioning as an alarm issuing portion, to issue an alarm to the operator with characters, signs, voice, light or vibrations.

As hereinbefore described, the ultrasound surgical apparatus 1A provides high operability. Furthermore, the ultrasound surgical apparatus 1 offers a superior level of safety.

Although the foregoing has described the treatment of the tissue 3 being outside of blood vessels, the ultrasound surgical apparatus 1A can also be used to treat the inside of a blood vessel, for example, to treat arteriosclerosis where a raised lump (plaque) is formed inside an artery. To remove plaques, rotablator apparatuses can also be used. A rotablator apparatus destroys a plaque by rapidly rotating a drill with diamond at a distal end portion of a guide wire inserted into a catheter of an endoscope apparatus. Although suction-type ultrasound surgical apparatuses offer a superior level of safety to the rotablator apparatuses, it is also not preferable that the distal end portion 31 come into contact with a blood vessel wall.

The ultrasound surgical apparatus 1A acquires information of a distance from the distal end portion 31, which is a treating portion, to an inner wall of a blood vessel, from the strength of the cavitation level signal corresponding to a state of cavitation generated in liquid between the distal end portion 31 and the inner wall of the blood vessel being the tissue 3, i.e., blood, and the control portion 23 controls the driving portion 22 on the basis of the distance. Therefore, the ultrasound surgical apparatus 1A provides superior operability and further safety.

### Third Embodiment

Next, an ultrasound surgical apparatus 1B of a third embodiment of the present invention will be described. It should be noted that components similar to those in the ultrasound surgical apparatus 1 of the first embodiment are denoted by the same reference numbers and a description thereof is omitted.

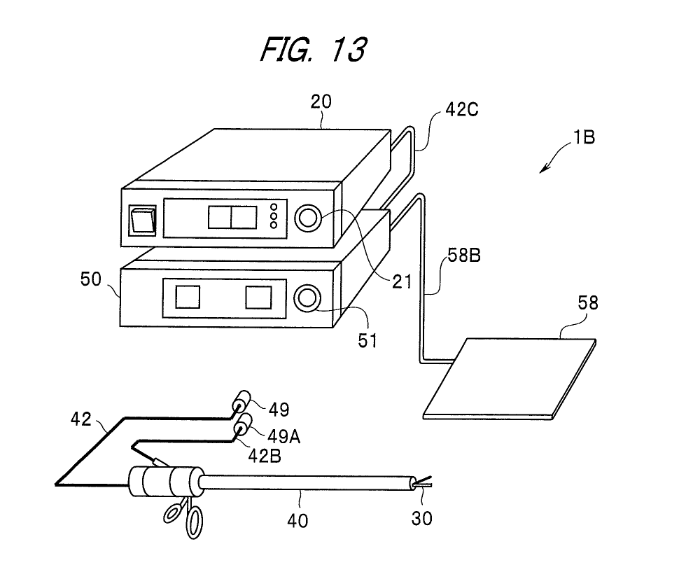

As shown in FIG. 13, the ultrasound surgical apparatus 1B is a scissors type ultrasound coagulating/cutting apparatus including an apparatus main body portion 20 and a handpiece 40B connected with the apparatus main body portion 20 through a cable 42. The ultrasound surgical apparatus 1B also includes a high frequency outputting portion 50 and a counter electrode 58 that runs high frequency current from a treating portion. That is, the handpiece 40B includes a probe 30 that can apply high frequency current to a distal end portion 31 being a treating portion and can carry out high frequency current treatment. The cylindrical column shaped probe 30 is provided in the handpiece 40B and an operation handle 43 that operates a grasping portion 45 at a distal end portion is provided at a proximal end portion.

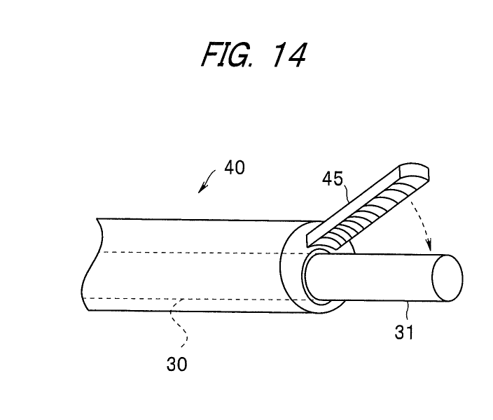

As shown in FIG. 14, the grasping portion 45 is moved toward the distal end portion 31 by an operator gripping the operation handle 43 (closing operation). The operator carries out friction heat treatment on a tissue 3 (not shown in FIG. 14) grasped between the grasping portion 45 and the distal end portion 31 using ultrasound vibrations.

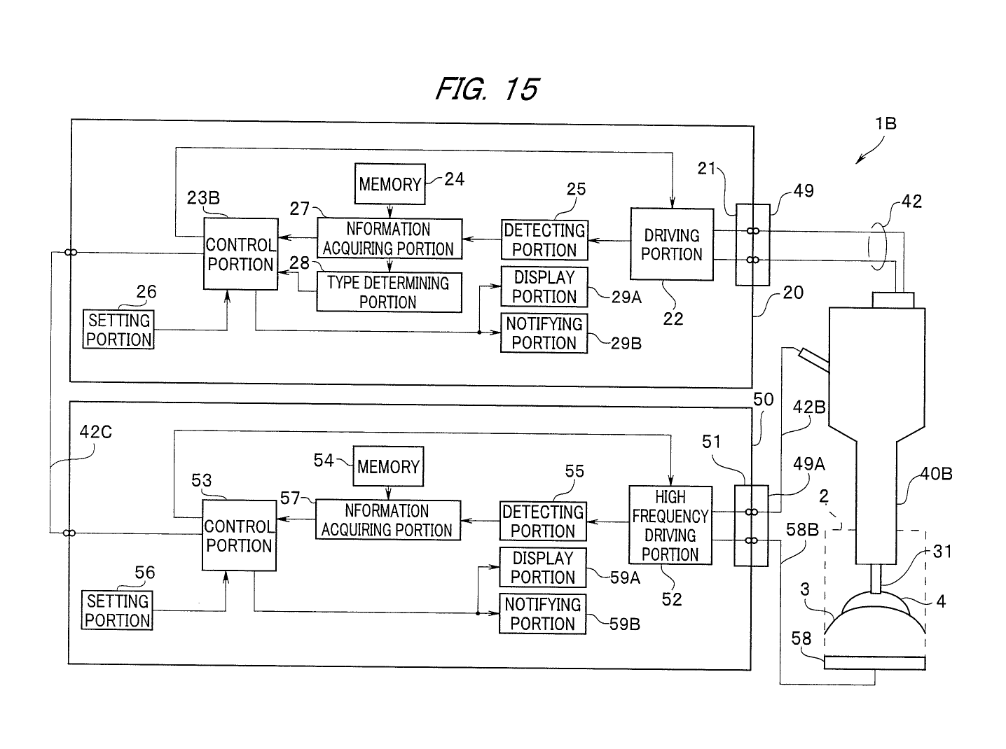

As shown in FIG. 15, the high frequency outputting portion 50 of the ultrasound surgical apparatus 1B includes components similar to those in the apparatus main body portion 20, acquires information of the tissue 3, and adjusts the strength of high frequency current running through the tissue 3 on the basis of the acquired information of the tissue 3.

That is, the high frequency outputting portion 50 includes a high frequency driving portion 52, a detecting portion 55, an information acquiring portion 57, memory 54, a control portion 53, a setting portion 56, a display portion 59A, and a notifying portion 59B. The control portion 53 of the high frequency outputting portion 50 is connected with a control portion 23B of the apparatus main body portion 20 via a cable 42C. The control portion 53 controls the high frequency outputting portion 50, while the control portion 23B controls the entire ultrasound surgical apparatus 1B including the high frequency outputting portion 50.

The high frequency current from the high frequency driving portion 52 is transferred to a cable 42B via a connector 49A of the handpiece 40B connected with a socket 51 of the high frequency outputting portion 50. Then, the high frequency current proceeds to the distal end portion 31, runs through the tissue 3, and reaches the counter electrode 58. The high frequency driving portion 52 is operated by setting of the setting portion 56 and control of the control portion 53. The detecting portion 55 detects, for example, electrical impedance of the tissue 3 between the distal end portion 31 and the counter electrode 58, and the information acquiring portion 57 acquires information of the tissue 3 on the basis of the impedance detected by the detecting portion 55 and data stored in the memory 54. The display portion 59A and the notifying portion 59B have functions similar to those of the display portion 29A and the notifying portion 29B described above. The control portion 53 controls the power of high frequency current outputted from the high frequency driving portion 52 on the basis of the information acquired by the information acquiring portion 57.

That is, the ultrasound surgical apparatus 1B has a treating function that uses ultrasound and a treating function that uses high frequency current, included in the known ultrasound surgical apparatuses, as well as the ultrasound surgical apparatus 1B has a function for acquiring information from the tissue 3 being treated, by using ultrasound and a function for acquiring information by using high frequency current.

It is noted that the information acquired by the information acquiring portion 57 of the high frequency outputting portion 50 may not be equal to the information acquired by the information acquiring portion 27 of the apparatus main body portion 20. For example, the information acquiring portion 57 of the high frequency outputting portion 50 acquires the information of the water content in the tissue 3 or type information about the type of the tissue 3, muscles, parenchymal organs or fatty tissues. Further, the information acquiring portion 57 may also acquire information such as an amount of energy applied to the tissue 3, a contact area between the tissue 3 and the distal end portion 31, and whether electricity is discharged or not. The information acquiring portion 27 can also acquire the information of a mechanical load such as a pressing force to the distal end portion 31. Thus, output to the probe 30 (ultrasound vibrations/high frequency current) can be controlled and the protection of the probe 30 is also enabled on the basis of the information, acquired by the information acquiring portion 27, of the stress applied to the distal end portion 31 by the tissue 3 and of whether the tissue 3 is in contact with the distal end portion 31.

For example, if the distal end portion 31 is not in contact with the tissue 3, the control portion 23B controls the amplitude of ultrasound vibrations to 30% of the maximum amplitude and high frequency output to 10 W, and if the distal end portion 31 is in contact with the tissue 3, the control portion 23B controls the amplitude of ultrasound vibrations to 70% of the maximum amplitude and high frequency output to 30 W.

If the water content in the tissue 3 is low, the information acquiring portion 27 can acquire accurate and more information, while if the water content in the tissue 3 is high, i.e., in the case of a wet condition, the information acquiring portion 57 can easily acquire information.

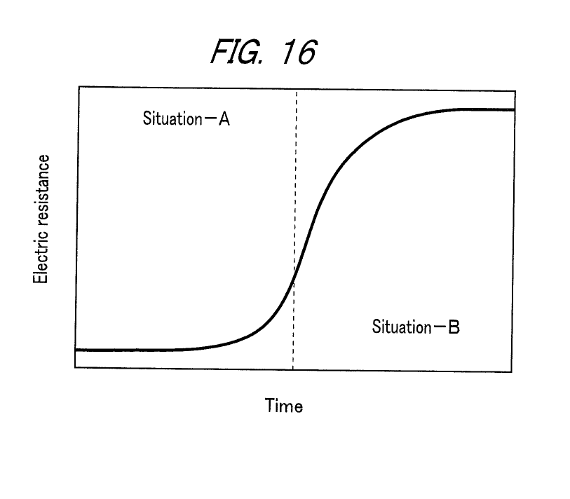

FIG. 16 shows a relationship in treatment between time course and the electric resistance of the tissue 3, i.e., the water content. As shown in FIG. 16, at the start of the treatment, the tissue 3 is in “situation A,” where the water content is high and the electric resistance is low. Then, as the treatment proceeds, the water in the tissue 3 is reduced, so that the water content is lowered and the electric resistance rises, resulting in “situation B.”

It is preferable that in “situation A,” the ultrasound surgical apparatus 1B acquire the information of the tissue 3 using the information acquiring portion 57 and in “situation B,” the ultrasound surgical apparatus 1B acquire the information of the tissue 3 using the information acquiring portion 27. That is, the control portion 23 controls at least any one of the driving portion 22 and the high frequency driving portion 52 on the basis of the information acquired by any one of the information acquiring portions 27 and 57 that easily acquires the information required for the treatment or can acquire accurate information. Therefore, the ultrasound surgical apparatus 1B has the advantages of the known ultrasound surgical apparatuses or the ultrasound surgical apparatus 1 as well as the ultrasound surgical apparatus 1B provides higher operability.

### Additional Notes

The transducer 35 of the ultrasound surgical apparatus to which the driving portion 22 intermittently supplies the driving signal may also be used as a sensor. For example, in a state where driving current is not applied, if the distal end portion 31 is brought into contact with a tissue, the transducer 35 receives pressing force. The electrical signal generated in the transducer 35 by the pressing force can be analyzed to obtain information of the tissue with which the distal end portion 31 is brought into contact, for example, hardness or viscoelasticity information. In addition, the transducer 35 is frequency swept at small amplitude and impedance characteristics of the tissue are analyzed, whereby the information of the tissue being treated by the distal end portion 31 can also be obtained. If the transducer 35 is used as a sensor and is frequency swept, it is preferable to damp the transducer 35 by shorting an electrode of the transducer 35 that is vibrating for treatment.

Alternatively, in a state where driving current is not applied, the transducer 35 can also detect the echo signal generated by reverberation of activation or burst sound of cavitation bubbles returning to the tissue 3. Then, the ultrasound surgical apparatus can acquire, as information of the tissue 3, boundary information between skeleton/internal organs and muscle tissues or information of acoustic impedance changing surfaces such as boundaries between different internal organs by analyzing the echo signal.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An ultrasound surgical apparatus comprising:
an ultrasound transducer that generates ultrasound vibrations;
a driving portion that supplies a driving signal to the ultrasound transducer;
a treating portion that is mechanically coupled with the ultrasound transducer and treats a living tissue through a liquid;
a detecting portion that detects a cavitation level signal corresponding to a state of cavitation generated in the liquid by ultrasound vibrations of the treating portion; and
an information acquiring portion that acquires information of the living tissue on the basis of the cavitation level signal.

2. The ultrasound surgical apparatus according to claim 1, further comprising
a control portion that controls a signal strength of the driving signal supplied by the driving portion in accordance with the information of the living tissue acquired by the information acquiring portion.

3. The ultrasound surgical apparatus according to claim 2, further comprising
an alarm issuing portion that issues an alarm in accordance with the information of the living tissue acquired by the information acquiring portion.

4. The ultrasound surgical apparatus according to claim 1, wherein
the cavitation level signal is a voltage signal, current signal, or impedance signal of the driving signal.

5. The ultrasound surgical apparatus according to claim 4, wherein
the cavitation level signal comprises components of frequencies other than driving frequencies of the driving signal.

6. The ultrasound surgical apparatus according to claim 5, wherein
the driving portion alternately supplies the ultrasound transducer with, as the driving signal, high output signal causing cavitation to be generated in the liquid and low output signal not causing cavitation to be generated in the liquid; and
the detecting portion detects the cavitation level signal while the low output signal is being supplied to the ultrasound transducer.

7. The ultrasound surgical apparatus according to claim 1, wherein
the driving portion intermittently supplies the driving signal to the ultrasound transducer; and
the detecting portion detects the cavitation level signal on the basis of output signal of the ultrasound transducer while the driving signal is not being supplied to the ultrasound transducer.

8. The ultrasound surgical apparatus according to claim 7, wherein
the information acquiring portion acquires information of the living tissue on the basis of a pace of attenuation of the cavitation level signal.

9. The ultrasound surgical apparatus according to claim 8, wherein
the information of the living tissue acquired by the information acquiring portion is hardness information of the living tissue.

10. The ultrasound surgical apparatus according to claim 9, wherein
the information acquiring portion decides that if the pace of attenuation of the cavitation level signal is higher, the living tissue is harder.

11. The ultrasound surgical apparatus according to claim 8, further comprising
a type determining portion that determines a type of the living tissue on the basis of the information of the living tissue acquired by the information acquiring portion.

12. The ultrasound surgical apparatus according to claim 1, wherein
the information of the living tissue acquired by the information acquiring portion is information of a distance from the living tissue to the treating portion or information of a water content in the living tissue.

13. The ultrasound surgical apparatus according to claim 12, wherein
the information acquiring portion decides that if a strength of the cavitation level signal is higher, the distance from the living tissue to the treating portion is shorter or the water content in the living tissue is higher.

14. The ultrasound surgical apparatus according to claim 13, further comprising:
a high frequency current driving portion that passes high frequency current through the treating portion;
a counter electrode through which the high frequency current from the treating portion is passed; and
a second information acquiring portion that acquires information of the living tissue on the basis of impedance between the treating portion and the counter electrode.

15. The ultrasound surgical apparatus according to claim 14, wherein
the control portion performs control in accordance with electric resistance of the living tissue on the basis of the information acquired by the information acquiring portion or the second information acquiring portion.

16. An ultrasound surgical apparatus comprising:
an ultrasound transducer that generates ultrasound vibrations;
a treating portion that is mechanically coupled with the ultrasound transducer and treats a living tissue through a liquid;
a detecting portion that detects a cavitation level signal corresponding to a state of cavitation generated in the liquid by ultrasound vibrations of the treating portion;
a driving portion that alternately supplies the ultrasound transducer with, as a driving signal, a high output signal causing cavitation to be generated in the liquid and a low output signal not causing cavitation to be generated in the liquid;
an information acquiring portion that acquires information of hardness of the living tissue on the basis of a pace of attenuation of the cavitation level signal detected by the detecting portion while the low output signal is being supplied to the ultrasound transducer; and
a control portion that controls a signal strength of the high output signal on the basis of the information acquired by the information acquiring portion.
